(19) United States
(12) Patent Application Publication
(10) Pub. No.: US 2006/0064017 A1
(43) Pub. Date: Mar. 23, 2006

(54) Hierarchical medical image view determination

(76) Inventors: Sriram Krishnan, Exton, PA (US); Jinbo Bi, Exton, PA (US); R. Bharat Rao, Berwyn, PA (US); Jonathan Stoeckel, Exton, PA (US); Matthew Eric Otey, Columbus, OH (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/231,593

(22) Filed: Sep. 21, 2005

Related U.S. Application Data
(60) Provisional application No. 60/611,865, filed on Sep. 21, 2004.

(51) Int. Cl. A61B 8/02

(52) U.S. Cl. 600/450

(57) ABSTRACT

A cardiac view of a medical ultrasound image is automatically identified. By grouping different views into sub-categories, a hierarchal classifier identifies the views. For example, apical views are distinguished from parasternal views. Specific types of apical or parasternal views are identified based on distinguishing between images of the geneses. Different features are used for classifying, such as gradients, functions of the gradients, statistics of an average frame of data from a clip or sequence of frames, or a number of edges along a given direction. The number of features used may be compressed, such as by classifying a plurality of features into a new feature. For example, alpha weights in a model of features and classes are determined and used as features for classification.

### RELATED APPLICATIONS

The present patent document claims the benefit of the filing date under 35 U.S.C. §119(e) of Provisional U.S. Patent Application Ser. No. 60/611,865, filed Sep. 21, 2004, the disclosure of which is hereby incorporated by reference.

### BACKGROUND

The present invention relates to classifying medical images. For example, a processor identifies cardiac views associated with medical ultrasound images.

In the field of medical imaging, various imaging modalities and systems generate medical images of anatomical structures of individuals for screening and evaluating medical conditions. These imaging systems include, for example, CT (computed tomography) imaging, MRI (magnetic resonance imaging), NM (nuclear magnetic) resonance imaging, X-ray systems, US (ultrasound) systems, PET (positron emission tomography) systems, or other systems. With ultrasound, sound waves propagate from a transducer towards a specific part of the body (the heart, for example). In MRI, gradient coils are used to “select” a part of the body where nuclear resonance is recorded. The part of the body targeted by the imaging modality usually corresponds to the area that the physician is interested in exploring. Each imaging modality may provide unique advantages over other modalities for screening and evaluating certain types of diseases, medical conditions or anatomical abnormalities, including, for example, cardiomyopathy, colonic polyps, aneurisms, lung nodules, calcification on heart or artery tissue, cancer micro calcifications or masses in breast tissue, and various other lesions or abnormalities.

Typically, physicians, clinicians, or radiologists manually review and evaluate medical images (X-ray films, prints, photographs, etc) to discern characteristic features of interest and detect, diagnose or otherwise identify potential medical conditions. Depending on the skill and knowledge of the reviewing physician, clinician, or radiologist, manual evaluation of medical images can result in misdiagnosed medical conditions due to simple human error. Furthermore, when the acquired medical images are of low diagnostic quality, it can be difficult for even a highly skilled reviewer to effectively evaluate such medical images and identify potential medical conditions.

Classifiers may automatically diagnose any abnormality to provide a diagnosis instead of, as a second opinion to or to assist a reviewer. Different views may assist diagnosis by any classifier. For example, apical four chamber, apical two chamber, parasternal long axis and parasternal short axis views assist diagnosis for cardiac function from ultrasound images. However, the different views have different characteristics. To classify the different views, different information may be important. However, identifying one view from another view may be difficult.

### BRIEF SUMMARY

By way of introduction, the preferred embodiments described below include methods, systems and computer readable media for identifying a cardiac view of a medical ultrasound image or classifying medical images. By grouping different views into sub-categories, a hierarchal classifier identifies the views. For example, apical views are distinguished from parasternal views. Specific types of apical or parasternal views are identified based on distinguishing between images of the geneses. Different features are used for classifying, such as gradients, functions of the gradients, statistics of an average frame of data from a clip or sequence of frames, or a number of edges along a given direction. The number of features used may be compressed, such as by classifying a plurality of features into a new feature. For example, alpha weights in a model of features and classes are determined and used as features for classification.

In a first aspect, a method is provided for identifying a cardiac view of a medical ultrasound image. With a processor, the medical ultrasound image is classified between any two or more of parasternal, apical, subcostal, suprasternal or unknown. With the processor, the cardiac view of the medical image is classified as a particular parasternal or apical view based on the classification as parasternal or apical, respectively.

In a second aspect, a system is provided for identifying a cardiac view of a medical ultrasound image. A memory is operable to store medical ultrasound data associated with the medical ultrasound image. A processor is operable to classify the medical ultrasound image between any two or more of subcostal, suprasternal, unknown, parasternal or apical from the medical ultrasound data, and is operable to classify the cardiac view of the medical image as a particular parasternal or apical view based on the classification as parasternal or apical, respectively.

In a third aspect, a computer readable storage media has stored therein data representing instructions executable by a programmed processor for identifying a cardiac view of a medical image. The instructions are for: first identifying the medical image as belonging to a specific generic class from two or more possible generic classes of subcostal view medical data, suprasternal view medical data, apical view medical data or parasternal view medical data; and second identifying the cardiac view based on the first identification.

In a fourth aspect, a computer readable storage media has stored therein data representing instructions executable by a programmed processor for identifying a cardiac view of a medical image. The instructions are for: extracting feature data from the medical image by determining one or more gradients from the medical ultrasound data, calculating a gradient sum, gradient ratio, gradient standard deviation or combinations thereof, determining a number of edges along at least a first dimension, determining a mean, standard deviation, statistical moment or combinations thereof of the intensities associated with the medical image, or combinations thereof, and classifying the cardiac view as a function of the feature data.

In a fifth aspect, a computer readable storage media has stored therein data representing instructions executable by a programmed processor for classifying a medical image. The instructions are for: extracting first feature data from the medical image; classifying at least second feature data from the first feature data; and classifying the medical image as a function of the second feature data with or without the first feature data.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for identifying medical images or image characteristics;

FIG. 2 is a flow chart diagram showing one embodiment of a method for hierarchal identification of medical image views;

FIGS. 3, 4 and 5 are scatter plots of gradient features for one example set of training information;

FIGS. 6 and 7 are example plots of intensity plots for identifying edges;

FIG. 8 shows four example histograms for deriving features; and

FIGS. 9-12 are plots of different classifier feature based performance for pixel intensity features.

### DETAILED DESCRIPTION OF THE DRAWINGS AND PRESENTLY PREFERRED EMBODIMENTS

Ultrasound images of the heart can be taken from many different angles. Efficient analysis of these images requires recognizing which position the heart is in so that cardiac structures can be identified. Four standard views include the apical two-chamber view, the apical four-chamber view, the parasternal long axis view, and the parasternal short axis view. Other views or windows include: apical five-chamber, parasternal long axis of the left ventricle, parasternal long axis of the right ventricle, parasternal long axis of the right ventricular outflow tract, parasternal short axis of the aortic valve, parasternal short axis of the mitral valve, parasternal short axis of the left ventricle, parasternal short axis of the cardiac apex, subcostal four chamber, subcostal long axis of inferior vena cava, suprasternal north long axis of the aorta, and suprasternal notch short axis of the aortic arch. To assist diagnosis, the views of cardiac ultrasound images are automatically classified. The view may be unknown, such as associated with a random transducer position or other not specifically defined view.

A hierarchical classifier classifies an unknown view as either apical, parasternal, subcostal, unknown or supracostal view, and then further classifies the view into one of the respective subclasses where the view is not unknown. Rather than one versus all or one versus one schemes to identify a class (e.g., distinguishing between from 15 views), multiple stages are applied for distinguishing different groups of classes from each other in a hierarchal approach (e.g., distinguish between a fewer number of classes at each level). By separating the classification, specific views may be more accurately identified. A specific view in any of the sub-classes may include an “unknown view” option, such as A2C, A4C and unknown options for apical sub-class. Single four or fifteen-class identification may be used in other embodiments.

Identification is a function of any combination of one or more features. For example, identification is a function of gradients, gradient functions, number of edges, or statistics of a frame of data averaged from a sequence of images. Features used for classification, whether for view identification or diagnosis based on a view, may be generated by compressing information in other features.

The classification outputs an absolute identification or a confidence or likelihood measure that the identified view is in a particular class. The results of view identification for a medical image can be used by other automated methods, such as abnormality detection, quality assessment methods, or other applications that provide automated diagnosis or therapy planning. The classifier provides feedback for current or future scanning, such as outputting a level of diagnostic quality of acquired images or whether errors occurred in the image acquisition process.

The classifier identifies views and/or conditions from one or more images. For example, views are identified from a sequence of ultrasound images associated with one or more heart beats. Images from other modalities may be alternatively or also included, such as CT, MRI or PET images. The classification is for views, conditions or both views and conditions. For example, the hierarchal classification is used to distinguish between different specific views. As another example, a model-based classifier compresses a number of features for view or condition classification.

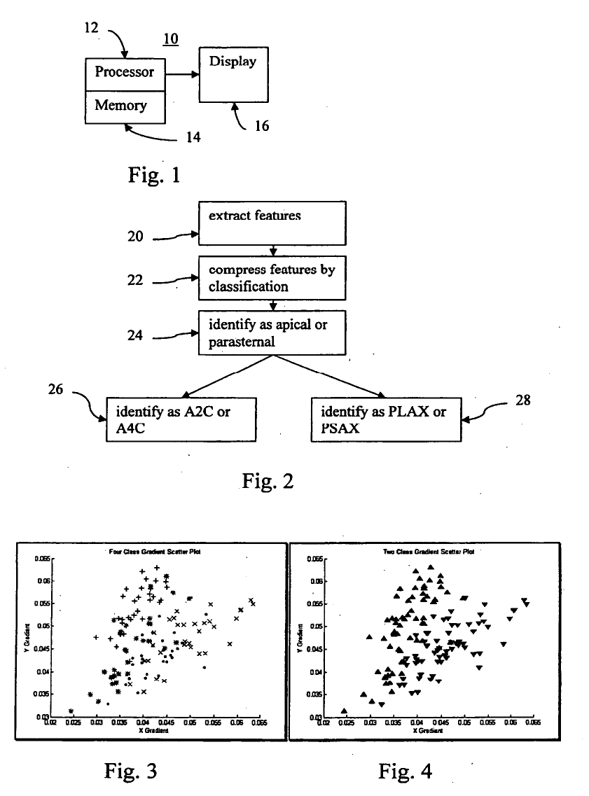

FIG. 1 shows a system 10 for identifying a cardiac view of a medical ultrasound image, for extracting features or for applying a classifier to medical images. The system 10 includes a processor 12, a memory 14 and a display 16. Additional, different or fewer components may be provided. The system 10 is a personal computer, workstation, medical diagnostic imaging system, network, or other now known or later developed system for identifying views or classifying medical images with a processor. For example, the system 10 is a computer aided diagnosis system. Automated assistance is provided to a physician, clinician or radiologist for identifying a view or classifying a state appropriate for given medical information, such as the records of a patient. Any view or abnormality diagnosis may be performed. The automated assistance is provided after subscription to a third party service, purchase of the system 10, purchase of software or payment of a usage fee.

The processor 12 is a general processor, digital signal processor, application specific integrated circuit, field programmable gate array, analog circuit, digital circuit, combinations thereof or other now known or later developed processor. The processor 12 is a single device or a plurality of distributed devices, such as processing implemented on a network or parallel processors. Any of various processing strategies may be used, such as multi-processing, multi-tasking, parallel processing or the like. The processor 12 is responsive to instructions stored as part of software, hardware, integrated circuits, film-ware, micro-code and the like.

The memory 14 is a computer readable storage media. Computer readable storage media include various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one embodiment, the instructions are stored on a removable media drive for reading by a medical diagnostic imaging system, a workstation networked with imaging systems or other programmed processor 12. An imaging system or work station uploads the instructions. In another embodiment, the instructions are stored in a remote location for transfer through a computer network or over telephone lines to the imaging system or workstation. In yet other embodiments, the instructions are stored within the imaging system on a hard drive, random access memory, cache memory, buffer, removable media or other device.

The instructions stored in the memory 14 control operation of the processor to classify, extract features, compress features and/or identifying a view, such as a cardiac view, of a medical image. For example, the instructions correspond to one or more classifiers or algorithms. In one embodiment, the instructions provide a hierarchical classifier using different classifiers or modules of Weka. Different class files from Weka may be independently addressed or run. Java components and script in bash implement the hierarchical classifier. Feature extraction is provided by Matlab code. Any format may be used for feature data, such as comma-separated-value (csv) format. The data is generated in such a way as to be used for leave-one-out cross-validation, such as by identifying different feature sets as corresponding with specific iterations or images. Other software with or without commercially available coding may be used.

The functions, acts or tasks illustrated in the figures or described herein are performed by the programmed processor 12 executing the instructions stored in the memory 14. The functions, acts or tasks are independent of the particular type of instructions set, storage media, processor or processing strategy and may be performed by software, hardware, integrated circuits, film-ware, micro-code and the like, operating alone or in combination.

Medical data is input to the processor 12 or the memory 14. The medical data is from one or more sources of patient information. For example, one or more medical images are input from ultrasound, MRI, nuclear medicine, x-ray, computer themography, angiography, and/or other now known or later developed imaging modeality. The imaging data is information that may be processed to generate an image, information previously processed to form an image, gray-scale values or color values. For example, ultrasound data formatted as frames of data associated with different two or three-dimensional scans at different times are stored. The frames of data are predetected, prescan converted or post scan converted data.

Additionally or alternatively, non-image medical data is input, such as clinical data collected over the course of a patient's treatment, patient history, family history, demographic information, billing code information, symptoms, age, or other indicators of likelihood related to the abnormality detection being performed. For example, whether a patient smokes, is diabetic, is male, has a history of cardiac problems, has high cholesterol, has high HDL, has a high systolic blood pressure or is old may indicate a likelihood of cardiac wall motion abnormality. The information is input by a user. Alternatively, the information is extracted automatically, such as shown in U.S. Pat. Nos. ______ (Publication No. 2003/0120458 (Ser. No. 10/287,055 filed on Nov. 4, 2002, entitled “Patient Data Mining”)) or ______ (Publication No. 2003/0120134 (Ser. No. 10/287,085, filed on Nov. 4, 2002, entitled “Patient Data Mining For Cardiology Screening”)), which are incorporated herein by reference. Information is automatically extracted from patient data records, such as both structured and un-structured records. Probability analysis may be performed as part of the extraction for verifying or eliminating any inconsistencies or errors. The system may automatically extract the information to provide missing data in a patient record. The processor 12 performs the extraction of information. Alternatively, other processors perform the extraction and input results, conclusions, probabilities or other data to the processors 12.

The processor 12 extracts features from images or other data. The features extracted may vary depending on the imaging modality, the supported clinical domains, and the methods implemented for providing automated decision support. Feature extraction may implement known segmentation and/or filtering methods for segmenting features or anatomies of interest by reference to known or anticipated image characteristics, such as edges, identifiable structures, boundaries, changes or transitions in colors or intensities, changes or transitions in spectrographic information, or other features using now known or later developed method. Feature data are obtained from a single image or from a plurality of images, such as motion of a particular point or the change in a particular feature across images.

The processor 12 uses extracted features to identify automatically the view of an acquired image. The processor 12 labels a medical image with respect to what view of the anatomy the medical image contains. By way of example, for cardiac ultrasound imaging, the American Society of Echocardiography (ASE) recommends using standard ultrasound views in B-mode to obtain sufficient cardiac image data—the apical two-chamber view (A2C), the apical four-chamber view (A4C), the apical long axis view (PLAX), the parasternal long axis view (PLAX), the parasternal short axis view (PSAX). Ultrasound images of the heart can be taken from various angles, but recognizing the position of the imaged heart (view) may enable identification of important cardiac structures. The processor 12 identifies an unknown cardiac image or sequence of images as one of the standard views and/or determines a confidence or likelihood measure for each possible view or a subset of views. The views may be non-standard or different standard views. The processor 12 may alternatively or additionally classify an image as having an abnormality.

The processor 12 is operable to apply different classifiers in a hierarchal model to the medical data. The classifiers are applied sequentially. The first classifier is operable to distinguish between two or more different classes, such as apical and parasternal classes. After the first classification or stage in the hierarchal model, a second classification or stage is performed. The second classifier is operable to distinguish between remaining groups of classes, such as two or four chamber views for apical data or long or short axis for parasternal data. The remaining more specific classes are a sub-set of the original possible classes without any more specific classes ruled out or assigned a probability in a previous stage. The classifier is free of considerations of whether the data is associated with any ruled out or already analyzed more generic classes. Given the different purposes or expected classes, the classifiers in each of the stages may be different, such as applying different thresholds, using different information, applying different weighting, trained from different datasets, or other differences.

In one embodiment, the processor 12 implements a model or classification system programmed with desired thresholds, filters or other indicators of class. For example, recommendations or other procedures provided by a medical institution, association, society or other group are reduced to a set of computer instructions. In response to patient information automatically determined by a processor or input by a user, the classifier implements the recommended procedure for identifying views. In an alternative embodiment, the system 10 is implemented using machine learning techniques, such as training a neural network using sets of training data obtained from a database of patient cases with known diagnosis. The system 10 learns to analyze patient data and output a view. The learning may be an ongoing process or be used to program a filter or other structure implemented by the processor 12 for later existing cases.

The processor 12 implements one or more techniques including a database query approach, a template processing approach, modeling and/or classification that utilize the extracted features to provide automated decision support functions, such as view identification. For example, database-querying methods search for similar labeled cases in a database. The extracted features are compared to the feature data of known cases in the database according to some metrics or criteria. As another example, template-based methods search for similar templates in a template database. Statistical techniques derive feature data for a template representative over a set of related cases. The extracted features from an image dataset under consideration are compared to the feature data for templates in the database. As another example, a learning engine and knowledge base implement a principle (machine) learning classification system. The learning engine includes methods for training or building one or more classifiers using training data from a database of previously labeled cases. It is to be understood that the term “classifiers” as used herein generally refers to various types of classifier frameworks, such as hierarchical classifiers, ensemble classifiers, or other now known or later developed classifiers. In addition, a classifier may include a multiplicity of classifiers that attempt to partition data into two groups and organized either organized hierarchically or run in parallel and then combined to find the best classification. Further, a classifier can include ensemble classifiers wherein a large number of classifiers (referred to as a “forest of classifiers”) all attempting to perform the same classification task are learned, but trained with different data, variables or parameters, and then combined to produce a final classification label. The classification methods implemented may be “black boxes” that are unable to explain their prediction to a user, such as classifiers built using neural networks. The classification methods may be “white boxes” that are in a human readable form, such as classifiers built using decision trees. In other embodiments, the classification models may be “gray boxes” that can partially explain how solutions are derived.

The display 16 is a CRT, monitor, flat panel, LCD, projector, printer or other now known or later developed display device for outputting determined information. For example, the processor 12 causes the display 16 at a local or remote location to output data indicating a view label of a medical image, extracted feature information, probability information, or other classification or identification. The output may be stored with or separate from the medical data.

FIG. 2 shows one embodiment of a method for identifying a cardiac view of a medical ultrasound image. Other methods for abnormality detection or feature extraction may be implemented without identifying a view. The method is implemented using the system 10 of FIG. 1 or a different system. Additional, different or fewer acts than shown in FIG. 2 may be provided in the same or different order. For example, acts 20 or 22 may not be performed. As another example, acts 24, 26, and/or 28 may not be performed.

The flow chart shown in FIG. 2 is for applying a hierarchal model to medical data for identifying cardiac views. The same or different hierarchal model may be used for detecting other views, such as other cardiac views or views associated with other organs or tissue.

Processor implementation of the hierarchal model may fully distinguish between all different possible views or may be truncated or end depending on the desired application. For example, medical practitioners may be only interested in whether the view associated with the patient record is apical or parasternal. The process may then terminate. The learning processes or other techniques for developing the classifiers may be based on the desired classes or views rather than the standard views.

Medical data representing one of at least three possible views is obtained. For example, the medical data is obtained automatically, through user input or a combination thereof for a particular patient or group of patients. In the example of FIG. 2, the medical data is for a patient being analyzed with respect to cardiac views. Cardiac ultrasound clips are classified into one of four categories, depending on which view of the heart the clip represents.

The images may clearly show the heart structure. In many images, the structure is less distinct. Ultrasound or other medical images may be noisy and have poor contrast. For example, an A2C clip may seem similar to a PSAX clip. With a small fan area and a difficult to see lower chamber, a round black spot in the middle may cause the A2C clip to be mistaken for a PSAX image. As another example, an A4C clip may seem similar to a PSAX clip. With a dim image having poor contrast, many of the chambers are hard to see, except for the left ventricle, making the image seem to be a PSAX image. As another example, horizontal streaks may cause misclassification as PLAX images. Tilted views may cause misclassification. Another problem is the fact that for the apical views, the four (or two) chambers are not very distinct. The apical views are often misclassified since the A4C views often show two large, distinct chambers, while the other two chambers are more difficult to see.

The data may be processed prior to classification or extraction of features. Machines of different vendors may output images with different characteristics, such as different image resolutions and different formats for presenting the ultrasound data on the screen. Even images coming from machines produced by a single vendor may have different fan sizes. The images or clip are interpolated, decimated, resampled or morphed to a constant size (e.g., 640 by 480) and the fan area is shifted to be the in the center of the image. A mask may limit undesired information. For example, a fan area associated with the ultrasound image is identified as disclosed in U.S. Pat. No. ______ (Publication No. ______ (application Ser. No. ______ (Attorney Docket No. 2004P17100US01), the disclosure of which is incorporated herein by reference. Other fan detection processes may be used, such as disclosed below. Alternatively, image information is provided in a standard field of view. As another alternative, the identification is performed for any sized field of view.

Intensities may be normalized prior to classification. First, the images of the clips are converted to grayscale by averaging over the color channels. Alternatively, color information is used to extract features. Some of the images may have poor contrast, reducing the distinction between the chambers and other areas of the image. Normalizing the grayscale intensities may allow better comparisons between images or resulting features. Linear normalization is of the form B=αA+β, where A is the original image and B is the normalized image. We let β=0, and set α=1/(U−L), where U is the value of the upper quartile of the image and L is the value of the lower quartile. A histogram of the intensities is formed. U and L are derived from the histogram, dividing by the interquartile range. Other values may be used to remove or reduce noise. Other normalization, such as minimum-maximum normalization may be used.

In act 20, feature data is extracted from the medical ultrasound data or other data for one or more medical images. The feature data is for one or more features for identifying views or other classification. Filtering, image processing, correlation, comparison, combination, or other functions extract the features from image or other medical data. Different features or combinations of features may be used for different identifications. Any now known or later developed features may be extracted.

In one example, one or more gradients are determined from one or more medical images. For example, three gradients are determined along three different dimensions. The dimensions are orthogonal with a third dimension being space or

xgrad = ygrad = 0;
for each frame {

find gradient in x-direction;
xsum = sum of magnitudes of all gradients in mask area;
xgrad = xgrad + xsum;
find gradient in y-direction;
ysum = sum of magnitudes of all gradients in mask area;
ygrad = ygrad + ysum;

}

time or are non-orthogonal, such as three dimensions being different angles within a two-dimensional plane. In one example, two dimensions (x, y) are perpendicular within a plane of each image within a sequence of images and the third dimension (z) is time within the sequence. The gradients in the x, y, and z directions provide the vertical and horizontal structure in the clips (x and y gradients) as well as the motion or changes between images in the clips (z gradients).

After masking or otherwise identifying the data representing the patient, the gradients are calculated. Gradients are determined for each image (e.g., frame of data) or for each sequence of images. The x and y gradients for each frame are determined as follows in one example:

xgrad = ygrad = 0;
for each frame {

find gradient in x-direction;
xsum = sum of magnitudes of all gradients in mask area;
xgrad = xgrad + xsum;
find gradient in y-direction;
ysum = sum of magnitudes of all gradients in mask area;
ygrad = ygrad + ysum;}

The x and y gradients are the sum of differences between each adjacent pair of values along the x and y dimensions. The gradients for each frame may be averaged, summed or otherwise combined to provide single x and y gradient values for each sequence. Other x and y gradient functions may be used.

The z gradients are found in a similar manner. The gradients between frames of data or images in the sequence are summed. The gradients are from each pixel location for each temporally adjacent pairs of images. Other z gradient functions may be used.

The gradient values are normalized by the number of voxels in the mask volume. For a single two-dimensional image, the number of voxels is the number of pixels. For a sequence of images, the number of voxels is the sum of the number of pixels for each image in the sequence.

In the example of cardiac ultrasound imaging to identify standard views, the four views show different structures. The gradients may discriminate between views. For example, the apical classes have a lot of vertical structure, the PLAX class has a lot of horizontal structure, and the PSAX class has a circular structure, resulting in different values for the x and y gradients. FIGS. 3 and 4 show scatter plots indicating separation between the classes using the x and y gradients in one example. The example is based on 129 training clips with 33 A2C, 33 A4C, 33 PLAX and 20 PSAX views. FIG. 3 shows all four classes (A2C, A4C, PLAX, and PSAX), and FIG. 4 shows the same plot generalized to the two super or generic classes—apical (downward facing triangles) and parasternal (upward facing triangles). FIG. 4 shows good separation between the apical and parasternal classes. FIG. 3 shows relatively good separation between the PLAX view (+) and the PSAX view (*). FIG. 3 shows less separation between the A2C (·) and A4C (x). However, the z gradients may provide more distinction between A2C and A4C views. There is different movement in the A2C and A4C views, such as two moving valves for A4C and one moving valve in A2C. The z gradient may distinguish between other views as well, such as between the PLAX class and the other classes.

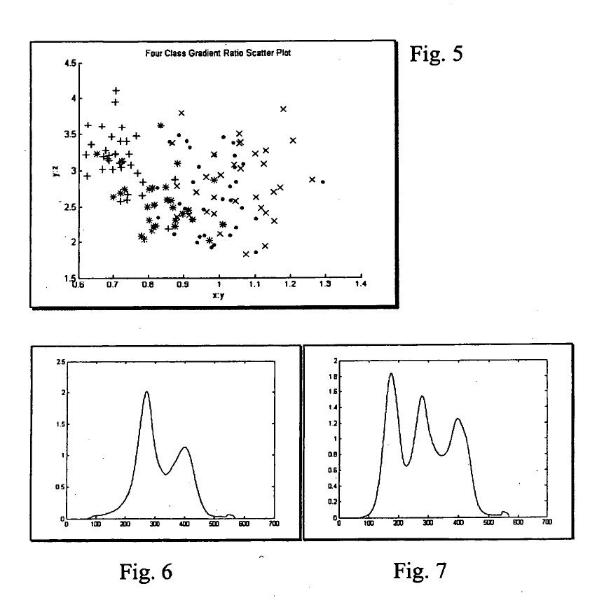

In another example, features are determined as a function of the gradients. Different functions may indicate class, such as view, with better separation than other functions. For example, XZ and YZ gradients features are calculated. The z-gradients throughout the sequence summed across all the frames of data, resulting in a two-dimensional image of z-gradients. The x and y gradients are calculated for the z-gradient image. The separations for the XZ and YZ gradients are similar to the separations for the X, Y and Z gradients. As another example, real gradients (Rx, Ry, and Rz) are computed without taking an absolute value. As yet another example, gradient sums (e.g., x+y, x+z, y+z) show decent separation between the apical and parasternal superclasses or generic views. As another example, gradient ratios (e.g., x:y, x:z, y:z) are computed by dividing one gradient feature by another. FIG. 5 shows a scatter plot of x:y versus y:z with fairly good separation. Another example is gradient standard deviations. For the x and y directions, the gradients for each frame of data are determined. The standard deviations of the gradients across a sequence are calculated. The standard deviation of the gradients within a frame or other statistical parameter may be calculated. For the z direction, the standard deviation of the magnitude of each voxel in the sequence is calculated.

In another example feature, a number of edges along one or more dimensions is determined. The number of horizontal and/or vertical edges or walls is
Take average of all frames to produce a single image matrix
Sum up over all rows of matrix
Normalize by the number of fan pixels in each column
Smooth this vector to remove peaks due to noise
xpeaks=the number of maxima in the vector
   counted in the images. Other directions may be used, including counts along curves or angled lines. The number of edges may discriminate between the A2C and A4C classes since the A2C images have only two walls while the A4C images have three walls.

Any now known or later developed function for counting the number of edges, walls, chambers, or other structures may be used. Different edge detection or motion detection processes may be used. In one embodiment, all of the frames in a sequence are averaged to produce a single image matrix. The data is summed over all rows of the matrix, providing a sum for each column. The sums are normalized by the number of pixels in each column. The resulting normalized sums may be smoothed to remove or reduce peaks due to noise. For example, a Gaussian, box car or other low pass filter is applied. The desired amount of smoothing may vary depending on the image quality. Too little smoothing may result in many peaks that do not correspond to walls in the image, and excessive smoothing may eliminate some peaks that do correspond to walls. By smoothing to provide an expected range of peaks, such as 2 or 3 peaks, the smoothing may be adapted to the image quality. FIGS. 6 and 7 show the smoothed magnitudes for A2C and A4C, respectively. There are two distinct peaks in the case of the A2C image, and three distinct peaks in the case of the A4C image. However, in each case there is a small peak on the right-hand side that may be removed by limiting the range of peak consideration and/or relative magnitude of the peaks. The feature is the number of maxima in the vector or along the dimension.

The number of peaks or valleys may provide little separation between the A2C and A4C classes. In the example set of 129 sequences, statistics for the number of x peaks in the A2C and A4C classes are provided as:

| | A2C | A4C |
|---|---|---|
| min | 1 | 3 |
| max | 9 | 6 |
| mean | 3.72 | 4.48 |
| median | 3 | 4 |

In other examples of extracting features, a mean, standard deviation, statistical moment, combinations thereof or other statistical features are extracted. The intensities associated with the medical image, an average medical image or through a sequence of medical images are determined. For example, the intensity distribution is characterized by averaging frames of data throughout a sequence of images and extracting the statistical parameter from the intensities of the averaged frame.

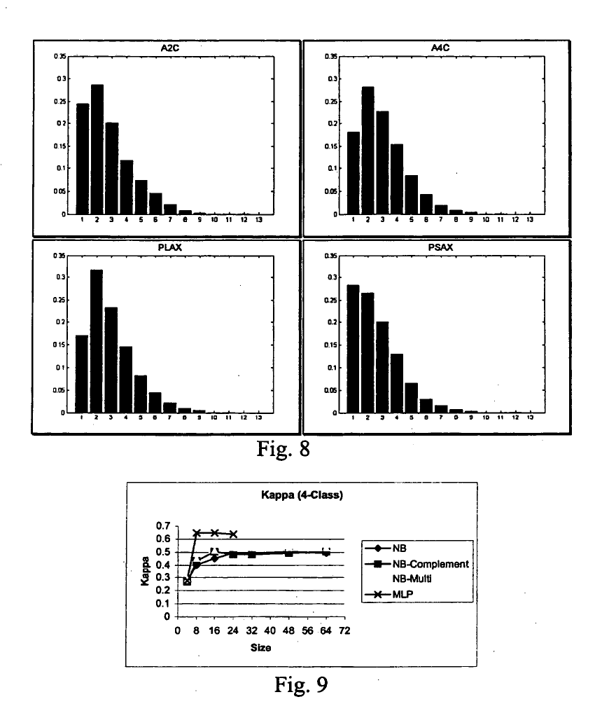

Other example features are extracted from pixel intensity histograms. The different classes or views have characteristic dark and light regions. The distribution of pixel intensities may reflect these differences. Frames of data in a sequence are averaged. Histograms for the average frame are generated with a desired bin width. FIG. 8 shows the average of all histograms in a class from the example training set of sequences. The average class histograms appear different from each other. From these histograms, it appears that the classes differ from one another in the values of the first four bins. Due to intra-class variance in these bins, poor separation may be provided. The variance may increase or decrease as a function of the width of the bins, intensity normalization, or where the class histograms simply do not represent the data. Variation of bin width or type of normalization may still result in variance. For views or data with less variance, a characteristic of the histograms may be a feature with desired separation. In one embodiment for the ultrasound cardiac example, the histograms are not used to extract features for classification.

Other example extracted features are raw pixel intensities. After normalization, the frames of data within a sequence are averaged across the sequence. So that there are a constant number of pixels for each clip, a universal mask is applied to the average frame. Where different sized images may be provided, the frames of the clip or the average frame are resized, such as by resampling, interpolation, decimation, morphing or filtering. The number of rows in the resized image (i.e. the new height) is denoted by r and the smoothing factor denoted by s. The resampling to provide r may result in a different s. The image is smoothed using a two-dimensional Gaussian filter with σ=sH/(2r), where H is the original height of the image. The result that two adjacent pixels in the resized image are smoothed by Gaussians that intersects at 1/s standard deviations away from their centers. The average frame may be filtered in other ways or in an additional process independent of r.

The number of resulting pixels is dependent on s and r. The resulting pixels may be used as features. The number of features affects the accuracy and speed of any classifier. The table below shows the number of features generated for a given r using a standard mask:

| r | # Features |
|---|---|
| 4 | 6 |
| 8 | 30 |
| 16 | 122 |
| 24 | 262 |
| 32 | 450 |
| 48 | 1016 |
| 64 | 1821 |

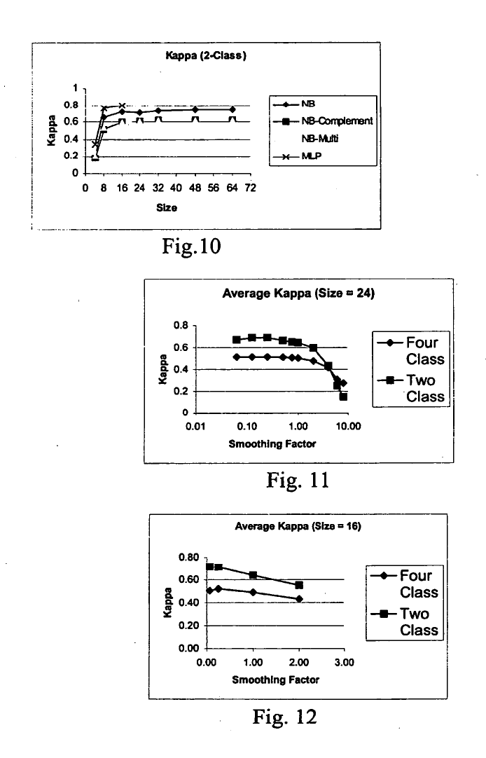

10-fold cross-validation using the raw pixel intensity features in Naïve Bayes Classifiers (NB) and a Multilayer Perceptron (MLP) using Weka provides different accuracy as a function of r. The accuracy is measured using the Kappa statistic, which is a measure of the significance of the number of matchings in two different labelings of a list. In one example using the 129 training sequences, s=1 and the height r of the image is varied. For classification on the four classes (A2C, A4C, PLAX, PSAX), FIG. 9 shows the Kappa value for different classifiers as a function of r. For two classes (apical, parasternal), FIG. 10 shows the Kappa value for different classifiers as a function of r. The MLP approach does not scale well for large numbers of attributes, so only partial results are shown. The accuracy levels at a value of r of about 16 to 24 rows. In another example using the 129 training sequences, the value of s varies for r equal to 16 and 24 rows. FIGS. 11 and 12 show Kappa averaged across all the classifiers used in FIGS. 9 and 10.

In general, more features (large height or r) provide greater accuracy. Even with less smoothing (smaller smoothing factor s), the accuracy remains relatively high. The raw pixel intensity feature may better distinguish between the two superclasses or generic views than between all four subclasses or specific views. The raw pixel intensity features may not be translation invariant. Structures may appear at different places in different images. Using a standard mask may be difficult where clips having small fan areas produce zero-valued features for the areas of the image that do not contain any part of an ultrasound, but are a part of the mask.

In act 22, one or more additional features are derived from a greater number of input features. The additional features are derived from subsets of the previous features by using an output of a classifier. Any classifier may be used. For example, a data set has n features per feature vector and c classes. Let Mᵢ be the model of the i^{th} class. In one example, Mᵢ is the average feature vector of the class, which infers that Mᵢ has n components. The additional feature vector is u. For classification, u is a weighted sum of all the Mᵢ's. This is represented as: u=α₁M₁+α₂M₂+ . . . +α_{c}M_{c} or in matrix format as: Mα=u where M is an n-by-c matrix where the i^{th} column vector is Mᵢ. α is limited as Σᵢα₁=1 and ∀i αᵢ≧0. A value for α that minimizes the squared error is determined. The additional feature vector u is then classified according to the index of the largest component of α.

α may represent a point in a c-dimensional “class space,” where each axis corresponds to one of the classes in the data set. There may be good separation between the classes in the class space. α may be used as the additional feature vector, replacing the u. This process may enhance the final classification by using the output of one classifier as the input to another in order to increase the accuracy.
T=Training data with only a subset of the features
T_{α}={ }
For all uεT {
Construct M from T−{U}
Solve Mα=u for α
T_{α}=T^{αu {α}}
}

Alpha features as the additional features ae derived from the image data using a leave-one-out approach. T=Training data with only a subset of the features. T_{α}={ }. For all uεT {Construct M from T−{u}, Solve Mα=u for α, and T_{α}=T_{α}u {α}}. The alpha features for testing data are derived by using a training set to construct M, and finding an a for each testing sample.

A large number of regular features are compressed into a fewer number of additional features. Features are compressed into just four (in the case of the four-class problem), two or other number of features. For example, alpha features are generated for both the two- and four-class problems using several different feature subsets, such as the raw pixel intensities for r=16, raw pixel intensities for r=24, and the x, y, and z gradients and/or other features. For example, alpha features are

| Naïve Bayes (Gradients Only) | | | | | Naïve Bayes (Alphas Only) | | | | |
|---|---|---|---|---|---|---|---|---|---|
| Real | a2c | a4c | plax | psax | Real | a2c | a4c | plax | psax |
| a2c | 19 | 7 | 1 | 6 | a2c | 19 | 8 | 0 | 6 |
| a4c | 8 | 23 | 0 | 2 | a4c | 8 | 24 | 0 | 1 |
| plax | 1 | 1 | 26 | 5 | plax | 0 | 0 | 29 | 5 |
| psax | 4 | 4 | 8 | 14 | psax | 4 | 2 | 7 | 17 |

| | |
|---|---|
| Accuracy = 63.6% | Accuracy = 69.0% |

derived from the 3-gradient (x, y, and z) feature subset for the two-class problem. The alpha features for the raw pixel intensity data provide reduction in data. For the case of r=24, the 262 attributes are reduced to 4, 2 or other number of features.

These replacement alpha features may provide good (or better) separation between classes than the original features, increasing accuracy. Confusion matrices with a basic Naïve Bayes or other classifier and leave-one-out cross-validation may indicate greater accuracy, such as greater accuracy for alpha features derived from three basic gradients than for accuracy for the three basic gradients. Misclassifications occur within the apical or parasternal superclasses. The misclassifications across the apical and parasternal superclasses tend to come from the parasternal short axis (PSAX) images.

The alpha features replace or are used in conjunction with the input features. The additional features are used with or without the input features for further classification. In one embodiment, some of the input features are not used for further classification and some are used.

All of the features may be used as inputs for classification. Other features may be used. Fewer features may be used. For example, the features used are the x, y and z gradient features, the gradient features derived as a function x, y and z gradient features, the count of structure features (e.g., wall or edge associated peak count), and the statistical features. Histograms or the raw pixel intensities are not directly used in this example embodiment, but may be in other embodiments. Four-class alpha features derived from the r=16 and r=24 raw pixel data sets with a smoothing factor of s=0.25, and alpha features derived from the three basic gradients are also used. In another example feature data set, the features to be used may be selected based on the training data. Attributes are removed in order to increase the value of the kappa statistic in the four-class problem. With a simple greedy heuristic, attributes are removed if they increased the value of kappa using a Naïve Bayes with Kernel Estimation or other classifier. The final reduced attribute data set contains 18 attributes: the alphas for r=16 raw pixel data, the alphas for the three-gradient data, the three basic gradients, the xz and yz gradients, the x:y and y:z gradient ratios, the z gradient standard deviation, the x peaks and the overall standard deviation. Other combinations may be used.

| Naïve Bayes with Kernel (FA) | | | | | Naïve Bayes with Kernel (RA) | | | | |
|---|---|---|---|---|---|---|---|---|---|
| Real | a2c | a4c | plax | psax | Real | a2c | a4c | plax | psax |
| a2c | 23 | 5 | 0 | 5 | a2c | 23 | 4 | 0 | 6 |
| a4c | 4 | 26 | 0 | 3 | a4c | 6 | 27 | 0 | 0 |
| plax | 0 | 2 | 28 | 3 | plax | 0 | 0 | 30 | 3 |
| psax | 4 | 1 | 6 | 19 | psax | 4 | 0 | 5 | 21 |

| | |
|---|---|
| Accuracy = 74.4% | Accuracy = 78.3% |

In acts 24, 26 and 28, the medical images are classified. One or more medical images are identified as belonging to a specific class or view. Any now known or later developed classifiers may be used. For example, Weka software provides implementations of many different classification algorithms. The NaYve Bayes Classifiers and/or Logistic Model Trees from the software are used. The Naïve Bayes Classifier (NB) is a simple probabilistic classifier. It assumes that all features are independent of each other. Thus, the probability that a feature vector X is in class Cᵢ is P(Cᵢ|X)=πⱼP(xⱼ|Cᵢ)P(Cᵢ). X is then assigned to the class to which it belongs with the highest probability. A normal distribution is usually assumed for the continuous-valued attributes of X, but a kernel estimator can be used instead. The Logistic Model Trees (LMT) is a classifier tree with logistic regression functions at the leaves.

The anomaly, view classification or other processes disclosed in U.S. Pat. Nos. ______ and ______ (Publication Nos. ______ and ______ (Application Nos. ______ and ______ (Attorney Docket Nos. 2003P09288US and 2004P04796US), the disclosures of which are incorporated herein by reference, may be used. In one embodiment, one or more classifiers are used to classify amongst all of the possible classes. For example, the NB, NB with a kernel estimator, and/or LMT classify image data as one of four standard cardiac ultrasound views. Other flat classifications may be used.

As an alternative to a flat classification, the processor applies a hierarchical classifier as shown in FIG. 2. In this example embodiment, there are three classifiers, one for each act to distinguish between parasternal and apical classes and sub-classes. Since misclassifications tend to be within the apical and parasternal classes, not across them, the hierarchal classification may avoid some misclassifications. In alternative embodiments, any two, three or all four of generic parasternal, apical, subcostal, and suprasternal generic classes and associated sub-classes are distinguished. While two layers of the hierarchy are shown, three or more layers may be used, such as distinguishing between apical and all other generic classes in one level, between parasternal and subcostal/suprasternal in another level and between subcostal and suprasternal in a fourth generic level. Unknown classification may be provided at any or all of the layers.

In act 22, a feature vector extracted from a medical image or sequence is classified into either the apical or the parasternal classes. The feature vector includes the various features extracted from the medical image data for the image, sequence of images or other data. Any classifier may be used, such as an LMT, NB with kernel estimation, or NB classifier to distinguish between the apical and parasternal views. In one embodiment, a processor implementing LMT performs act 22 to distinguish between apical and parasternal views.

In acts 24 and 26, the feature vector is further classified into the respective subclasses or specific views. The same or different features of the feature vector are used in acts 24 or 26. The specific views are identified based on and after the identification of act 22. If the medical data is associated with parasternal views, then act 24 is performed, not act 26. In act 24, the medical data is associated with a specific view, such as PLAX or PSAX. If the medical data is associated with apical views, then act 26 is performed, not act 24. In act 26, the medical data is associated with a specific view, such as A2C or A4C. Alternatively, both acts 24 and 26 are performed for providing probability information. The result of act 22 is used to set, at least in part, the probability.

The same or different classifier is applied in acts 24 and 26. One or both classifiers may be the same or different from the classifier applied in act 22. The algorithms of the classifiers identify the view. Given the different possible outputs of the three acts 22, 24 and 26, the different algorithms are applied even using the same classifiers. In one embodiment, a kernel estimator-based Naïve Bayes Classifier to distinguish between the subclasses in each of acts 24 and 26. Other classifiers may be used, such as a NB without kernel estimation or LMT. Different classifiers may be used for different types of data or features.

One or more classifiers alternatively identify an anomaly, such as a tumor, rather than or in addition to classifying a view. The processor implements additional classifiers to identify a state associated with medical data. Image analysis may be performed with a processor or automatically for identifying other characteristics associated with the medical data. For example, ultrasound images are analyzed to determine wall motion, wall thickening, wall timing and/or volume change associated with a heart or myocardial wall of the heart.

The classifications are performed with neural network, filter, algorithm, or other now-known or later developed classifier or classification technique. The classifier is configured or trained for distinguishing between the desired groups of states. For example, the classification disclosed in U.S. Pat. No. ______ (Publication No. 2005/0059876 (application Ser. No. 10/876,803)), the disclosure of which is incorporated herein by reference, is used. The inputs are received directly from a user, determined automatically, or determined by a processor in response to or with assistance from user input.

The system of FIG. 1 or other system implementing FIG. 2 is sold for classifying views. Alternatively, a service is provided for classifying the views. Hospitals, doctors, clinicians, radiologists or others submit the medical data for classification by an operator of the system. A subscription fee or a service charge is paid to obtain results. The classifiers may be provided with purchase of an imaging system or software package for a workstation or imaging system.

In one embodiment, the image information is in a standard format or the scan information is distinguished from other information in the images. Alternatively and as discussed above, the scan information representing the tissue of the patient is identified automatically. For ultrasound data, the scan information is circular, rectangular or fan shaped (e.g., sector or Vector® format). To derive features for classification, the fan or scan area is detected, and a mask is created to remove regions of the image associated with other information.

In one approach, the upper edges of an ultrasound fan are detected, and parameters of lines that fit these edges are calculated. The bottom of the fan is then detected from a histogram mapped as a function of radius from an intersection of the upper edges.
1. Let C=Ultrasound Clip
2. Cflat=Average C across-all frames
3. Cbw=Average Cflat across color channels
4. Csmooth=Cbw smoothed using a Gaussian filter
5. Find all connected regions of Csmooth
6. Select the region in the center of the Csmooth
7. Erode the borders of Csmooth
8. Mask=Csmooth

In another approach, the largest connected region in the image is identified as the fan area. C is an ultrasound clip. Cflat is an average C across all frames. Cbw is an average Cflat across color channels (i.e., convert color information into gray scale). Csmooth is Cbw smoothed using a Gaussian filter. All the connected regions of Csmooth are found. The region in the center of the Csmooth is selected. The borders of Csmooth are eroded, filtered or clipped to remove rough edges. The remaining borders define the Boolean mask. Due to erosion, the mask is slightly smaller than the actual fan area. The mask derived from one image in a sequence is applied to all of the images in the sequence.

The mask may be refined. Masks are determined for two or more images of the sequence. All of the masks are summed. A threshold is applied to the resulting sum, such as removing regions that appear in less than 80 or other number of masks. This allows holes in the individual masks to fill in.

In a different refinement, the largest connected region, W, in the image and an area S defined by identification of the upper edges are separately calculated. Most of the points in W should also be in S. A circular area C centered at the apex of S such that the area S∩C contains the maximum possible number of points in W while minimizing the number of points in ˜W is found. C defines a sector that encompasses as much of W as possible without including too many points that are not in W (i.e. points not belonging to the fan area). To find this sector, a cost function, Cost=|S∩C∩W|+|S∩˜(W∩S)|−|W∩S∩C| or other function, is minimized. The first term in this expression is the number of points in the sector not belonging to largest connected region. The second term is the number of points that belong to both the largest connected region and the triangle, but do not belong to the sector. The last term is the number of points in the largest connected region contained within the sector. After a sector has been found that minimizes this cost, the sector is eroded to prevent edge effects and is kept as the final mask for this image.

For larger fan areas clipped on the display (i.e., not a true fan), the best sector may also stretch out of the bounds of the image. To compensate for this, the radius of the circle C is limited to be no more than the height of the image. A further problem arises when the region W contains points which are not a part of the true fan area (e.g. diagnostic information along the bottom of the image). For example, diagnostic information touches or is superimposed on the fan area. The information may remain in the image or is otherwise isolated, such as by pattern matching letter, numeral or symbols.

Two or more mask generation approaches may be used. The results are combined, such as finding a closest fit, averaging or performing an “and” operation.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for identifying a cardiac view of a medical ultrasound image, the method comprising:
classifying, with a processor, the medical ultrasound image between any two or more of subcostal, suprasternal, parasternal, apical or unknown; and
classifying, with the processor, the cardiac view of the medical image as a particular subcostal, suprasternal, parasternal or apical view based on the classification as subcostal, suprasternal, parasternal or apical, respectively.

2. The method of claim 1 wherein classifying the cardiac view of the medical image comprises classifying as apical two chamber or apical four chamber for apical or as parasternal long axis or parasternal short axis for parasternal.

3. The method of claim 1 wherein classifying the cardiac view comprises applying different algorithms based on the classification of parasternal or apical.

4. The method of claim 1 wherein classifying the medical ultrasound image as subcostal, suprasternal, parasternal or apical comprises applying a classifier tree with logistic regression functions, and wherein classifying the cardiac view of the medical image as a particular parasternal or apical view comprises applying a Naïve Bayes Classifier.

5. The method of claim 1 further comprising:
extracting feature data from the medical ultrasound image;
wherein either or both of the classifying acts are performed as a function of the feature data.

6. The method of claim 5 wherein extracting the feature data comprises:
determining one or more gradients from the medical image;
calculating a gradient sum, gradient ratio, gradient standard deviation or combinations thereof; or
both determining and calculating.

7. The method of claim 5 wherein extracting the feature data comprises determining a number of edges along at least a first dimension.

8. The method of claim 5 wherein extracting the feature data comprises determining a mean, standard deviation, statistical moment or combinations thereof of the intensities associated with the medical image.

9. The method of claim 5 wherein extracting the feature data comprises classifying at least one additional feature from a plurality of input features, the feature data including the at least one additional feature with or without the input features.

10. A system for identifying a cardiac view of a medical ultrasound image, the method comprising:
a memory operable to store medical ultrasound data associated with the medical ultrasound image;
a processor operable to classify the medical ultrasound image between any two or more of subcostal, suprasternal, parasternal, apical or unknown from the medical ultrasound data, and operable to classify the cardiac view of the medical image as a particular subcostal, suprasternal, parasternal or apical view based on the classification as subcostal, suprasternal, parasternal or apical, respectively.

11. The system of claim 10 wherein the processor is operable to classify the cardiac view of the medical image as apical two chamber or apical four chamber for apical or as parasternal long axis or parasternal short axis for parasternal.

12. The system of claim 10 wherein the processor is a single device or a plurality of distributed devices, the processor further operable to extract feature data from the medical ultrasound data, wherein either or both of the classifying acts are performed as a function of the feature data.

13. The system of claim 12 wherein the processor is operable to extract the feature data by:
determining one or more gradients from the medical ultrasound data;
calculating a gradient sum, gradient ratio, gradient standard deviation or combinations thereof;
determining a number of edges along at least a first dimension;
determining a mean, standard deviation, statistical moment or combinations thereof of the intensities associated with the medical image; or
combinations thereof.

14. The system of claim 12 wherein the processor is operable to extract the feature data by classifying at least one additional feature from a plurality of input features, the feature data including the at least one additional feature with or without the input features.

15. In a computer readable storage media having stored therein data representing instructions executable by a programmed processor for identifying a cardiac view of a medical image, the storage media comprising instructions for:
first identifying the medical image as belonging to a specific generic class from two or more possible generic classes of subcostal view medical data, suprasternal view medical data, apical view medical data or parasternal view medical data;
second identifying the cardiac view based on the first identification.

16. The instructions of claim 15 wherein first identifying comprises classifying the medical image as the apical view medical data or as the parasternal view medical data, and wherein second identifying the cardiac view comprises classifying, after first identifying, the apical view medical data as apical two chamber or apical four chamber or classifying the parasternal view medical data as parasternal long axis or parasternal short axis.

17. The instructions of claim 15 wherein second identifying comprises identifying with a first algorithm based on the identification of the medical image as apical view medical data and identifying with a second algorithm different than the first algorithm based on the identification of the medical ultrasound image as parasternal view medical data.

18. The instructions of claim 15 wherein first identifying comprises applying a classifier tree with logistic regression functions, and wherein second identifying comprises applying a Naïve Bayes Classifier.

19. The instructions of claim 15 further comprising:
extracting feature data from data for the medical image;
wherein either or both of the first and second identifying acts are performed as a function of at least some of the feature data.

20. The instructions of claim 19 wherein extracting comprises determining a first gradient along a first dimension, a second gradient along a different dimension, a third gradient along another different dimension, a gradient parameter that is a function of the first parameter, second parameter, third parameter, or combinations thereof, or combinations thereof.

21. The instructions of claim 19 wherein extracting the feature data comprises determining a number of edges along at least a first dimension.

22. The instructions of claim 19 wherein extracting the feature data comprises determining a mean, standard deviation, statistical moment or combinations thereof of the intensities associated with the medical image.

23. The instructions of claim 19 wherein extracting the feature data comprises classifying at least one additional feature from a plurality of input features, the feature data including the at least one additional feature with or without the input features.

24. In a computer readable storage media having stored therein data representing instructions executable by a programmed processor for identifying a cardiac view of a medical image, the storage media comprising instructions for:
extracting feature data from the medical image by:
determining one or more gradients from the medical ultrasound data;
calculating a gradient sum, gradient ratio, gradient standard deviation or combinations thereof;
determining a number of edges along at least a first dimension;
determining a mean, standard deviation, statistical moment or combinations thereof of the intensities associated with the medical image; or
combinations thereof; and
classifying the cardiac view as a function of the feature data.

25. In a computer readable storage media having stored therein data representing instructions executable by a programmed processor for classifying a medical image, the storage media comprising instructions for:
extracting first feature data from the medical image;
classifying at least second feature data from the first feature data;
classifying the medical image as a function of the second feature data with or without the first feature data.

26. The instructions of claim 25 wherein classifying the at least second feature data comprises:
finding a weight value minimizing an error of a matrix including the first feature data as a function of classes;
selecting the weight value as the second feature data.
